(19) United States
(12) Patent Application Publication
(10) Pub. No.: US 2024/0197301 A1
(43) Pub. Date: Jun. 20, 2024

(54) ACCESSING, IMPLANT ANCHORING, AND VISUALIZATION DURING CORONARY ARTERY BYPASS GRAFT

(71) Applicant: EDWARDS LIFESCIENCES CORPORATION, Irvine, CA (US)

(72) Inventors: Taylor Jacob Scheinblum, Newport Beach, CA (US); David Robert Landon, Huntington Beach, CA (US); Kevin M. Stewart, Powell, TN (US); Matthew Michael Becerra, Lake Forest, CA (US); Alexander H. Cooper, Costa Mesa, CA (US); Wei Gan, Irvine, CA (US)

(21) Appl. No.: 18/591,604

(22) Filed: Feb. 29, 2024

Related U.S. Application Data
(63) Continuation of application No. PCT/US2022/040606, filed on Aug. 17, 2022.
(60) Provisional application No. 63/239,283, filed on Aug. 31, 2021.

(51) Int. Cl. A61B 17/00; A61B 17/11

(52) U.S. Cl. CPC A61B 17/00234; A61B 17/11; A61B 2017/00252; A61B 2017/1107; A61B 2017/1139

(57) ABSTRACT

Traditional CABG procedures are performed by accessing the chest cavity and stopping the heart. However, off-pump CABG is becoming more wide-spread in use. Transcatheter CABG procedures are particularly advantageous and thus may become prevalent. A transcatheter CABG procedure can advantageously reduce complications, procedure time, and recovery time as well as providing benefits of being an off-pump, beating heart procedure. Accordingly, disclosed herein are several mechanisms and methods for accessing, implant anchoring, and visualization during a transcatheter CABG procedure.

### CROSS-REFERENCE TO RELATED APPLICATION

This application is a continuation of International Patent Application No. PCT/US2022/040606, filed on Aug. 17, 2022 and entitled ACCESSING, IMPLANT ANCHORING, AND VISUALIZATION DURING CORONARY ARTERY BYPASS GRAFT, which claims priority to U.S. Provisional Application No. 63/239,283, filed on Aug. 31, 2021 and entitled ACCESSING, IMPLANT ANCHORING, AND VISUALIZATION DURING CORONARY ARTERY BYPASS GRAFT, the complete disclosures of which are hereby incorporated by reference in their entireties for all purposes.

### BACKGROUND

### Field

The present disclosure relates to the field of medical devices and procedures for coronary artery bypass grafting.

### Description of the Related Art

Coronary artery bypass grafting (CABG) is a type of surgery that improves blood flow to the heart. It is the most common type of open-heart surgery in the United States. CABG is appropriate for people who have severe coronary heart disease (CHD) or coronary artery disease (CAD) which is the narrowing of the coronary arteries—the blood vessels that supply oxygen and nutrients to the heart muscle. CAD is caused by a build-up of fatty material within the walls of the arteries. This build-up narrows the inside of the arteries, limiting the supply of oxygen-rich blood to the heart muscle.

During typical CABG, a healthy artery or vein from the body is connected, or grafted, to the blocked coronary artery. The grafted artery or vein bypasses the blocked portion of the coronary artery. This creates a new passage, and oxygen-rich blood is routed around the blockage to the heart muscle.

### SUMMARY

In some aspects, the present disclosure provides methods and/or devices for cardiac artery bypass grafting (CABG) procedures that include accessing a targeted vessel, anchoring an implant, and visualizing the targeted vessel. The various methods and devices described herein for accessing the targeted vessel can be combined with any of the compatible methods and devices described herein for anchoring the implant. Similarly, the various methods and devices described herein for anchoring the implant can be combined with any of the compatible methods and devices described herein for visualizing the targeted vessel. Furthermore, the various methods and devices described herein for accessing the targeted vessel can be combined with any of the compatible methods and devices described herein for visualizing the targeted vessel.

In some implementations, accessing the targeted vessel includes a transfemoral artery catheter in the aorta that punctures into the right atrium to access the right coronary artery. In some implementations, accessing the targeted vessel includes a transfemoral artery catheter in the aorta that punctures into the pericardial or chest cavity space to access the right or left coronary artery. In some implementations, accessing the targeted vessel includes puncturing through the left ventricular or atrial wall into the targeted vessel. In some implementations, accessing the targeted vessel includes accessing an adjacent artery or an artery from the opposite side of the heart and then accessing the targeted vessel from that artery. In some implementations, accessing the targeted vessel includes accessing the targeted vessel upstream of the blockage, puncturing through the targeted vessel, and reentering the targeted vessel downstream of the blockage.

In some implementations, anchoring the implant includes the use of wires, hooks, barbs, or the like at the end of a new vessel used to create the bypass or a shunt in the targeted vessel upstream of the blockage. In some implementations, visualizing the targeted vessel includes using a fiber optic that is part of the catheter, using an intravascular ultrasound (IVUS) catheter, using a direct view camera, or visualization of angiogram dye on fluoroscopy.

In some implementations, a coronary artery is accessed by directly puncturing through the left ventricle or left atrium wall into the artery and placing a shunt to cause port to remain open. In some implementations, the present disclosure provides for shunting between coronary arteries (e.g., from the left coronary artery to the right coronary artery). In some implementations, the present disclosure provides for enabling stable access to a targeted vessel using a suction-cup mechanism on a catheter. In some implementations, the present disclosure provides for inserting a line into a wall near a blocked vessel to seed angiogenesis.

In some implementations, the present disclosure provides for anchoring a stent within a targeted vessel upstream of a blockage and attaching the stent to a CABG line or shunt that is anchored to the targeted vessel downstream of the blockage.

In some implementations, the coronary artery can be accessed up to a blockage. In some implementations, after accessing the coronary artery, the blockage can be navigated around to puncture the targeted vessel downstream of the blockage. In some implementations, a balloon can be inflated to provide stability for the catheter to puncture out of the vessel, and an electrocautery catheter with a deflectable tip can be navigated around the blockage and re-access the vessel, thereby establishing a path for implant delivery. In some implementations, a coil-shaped catheter with electrocautery tip can be fed out of the vessel near the blockage, and the coil shape can provide stability following the vessel past the blockage where it can re-enter the vessel and leave a guidewire path for implant delivery. In some implementations, the implant itself can be coil shaped with a central lumen, acting as its own anchor, and providing shunting through the ventricular wall.

In some implementations, the techniques described herein relate to a method for performing cardiac artery bypass grafting, the method including: navigating a transfemoral artery catheter to an aorta of a patient; puncturing through the aorta with the transfemoral artery catheter; navigating the transfemoral artery catheter to a targeted vessel with a blockage; and implanting a shunt to bypass the blockage.

In some implementations, the techniques described herein relate to a method, wherein puncturing through the aorta includes puncturing into a right atrium to access a right coronary artery. In some implementations, the techniques described herein relate to a method, wherein puncturing through the aorta includes puncturing into a pericardial space to access a right coronary artery or a left coronary artery. In some implementations, the techniques described herein relate to a method, wherein puncturing through the aorta includes puncturing an aortic arch of the aorta. In some implementations, the techniques described herein relate to a method, wherein puncturing through the aorta includes puncturing an aortic root of the aorta. In some implementations, the techniques described herein relate to a method further including puncturing through a ventricular wall to access the targeted vessel. In some implementations, the techniques described herein relate to a method further including puncturing into a ventricle from a pericardial space prior to puncturing through the ventricular wall to access the targeted vessel.

In some implementations, the techniques described herein relate to a method further including advancing a sealing device to a puncture location including a location where the transfemoral artery catheter punctures through the aorta. In some implementations, the techniques described herein relate to a method, wherein the sealing device is secured to a portion of the transfemoral artery catheter so that advancing the transfemoral artery catheter causes the sealing device to contact material around the puncture location to achieve hemostasis. In some implementations, the techniques described herein relate to a method, wherein the sealing device is attached to the aorta prior to introducing the transfemoral artery catheter through the aorta.

In some implementations, the techniques described herein relate to a method for performing cardiac artery bypass grafting, the method including: navigating a catheter to a ventricle of a patient; puncturing through a ventricular wall into a targeted vessel with a blockage; and implanting a shunt between the ventricle and the targeted vessel such that the shunt is downstream of the blockage and the shunt enables passage of blood from the ventricle to the targeted vessel.

In some implementations, the techniques described herein relate to a method, wherein the shunt includes a coil-shaped anchor with a central lumen. In some implementations, the techniques described herein relate to a method, wherein the shunt further includes a plurality of perforations in the shunt to facilitate transfer of blood.

In some implementations, the techniques described herein relate to a method for performing cardiac artery bypass grafting, the method including: navigating a catheter to a first coronary artery of a patient; puncturing through the first coronary artery of the patient; navigating the catheter to a second coronary artery of the patient, the second coronary artery including a blockage; and implanting a shunt from the first coronary artery of the patient to the second coronary artery of the patient, the shunt being anchored to the second coronary artery of the patient downstream of the blockage.

In some implementations, the techniques described herein relate to a method for performing cardiac artery bypass grafting, the method including: navigating a catheter within a targeted vessel of a patient, the targeted vessel including a blockage, the catheter navigated to a position upstream of the blockage; puncturing through the targeted vessel with the catheter; navigating the catheter outside the targeted vessel to a point downstream of the blockage; and re-entering the targeted vessel with the catheter downstream of the blockage.

In some implementations, the techniques described herein relate to a method, wherein the catheter includes a coiled configuration to wrap around the targeted vessel. In some implementations, the techniques described herein relate to a method, wherein the catheter includes a deflectable tip. In some implementations, the techniques described herein relate to a method, wherein the catheter includes an electrocautery tip. In some implementations, the techniques described herein relate to a method further including inflating a balloon to stabilize the catheter for puncturing through the targeted vessel.

In some implementations, the techniques described herein relate to a method for performing cardiac artery bypass grafting, the method including: navigating a catheter to a targeted vessel of a patient, the targeted vessel including a blockage, the catheter navigated to a position downstream of the blockage; anchoring an implant to the targeted vessel, the implant including anchors at a distal end of the implant configured to secure to the targeted vessel; and creating a passageway from the implant to the targeted vessel to enable blood flow in the targeted vessel downstream of the blockage.

In some implementations, the techniques described herein relate to a method, wherein the passageway is created after anchoring the implant to the targeted vessel. In some implementations, the techniques described herein relate to a method, wherein the passageway is created prior to anchoring the implant to the targeted vessel. In some implementations, the techniques described herein relate to a method, wherein creating the passageway includes implanting a stent within the targeted vessel upstream of the blockage and anchoring a distal end of a shunt downstream of the blockage, the shunt providing the passageway between the stent and the targeted vessel at the distal end of the shunt. In some implementations, the techniques described herein relate to a method, wherein the distal end of the shunt includes a helical anchored that is anchored into muscle tissue at the targeted vessel.

In some implementations, the techniques described herein relate to a method further including directly visualizing the targeted vessel with a fiber optic cable that is part of the catheter. In some implementations, the techniques described herein relate to a method further including visualizing angiogram dye on fluoroscopy responsive to a tip of the catheter being inserted into the targeted vessel.

In some implementations, the techniques described herein relate to a method for performing cardiac artery bypass grafting, the method including: navigating a catheter to a targeted vessel of a patient, the targeted vessel including a blockage, the catheter navigated to a position downstream of the blockage; anchoring a supply line that is partially embedded in a cardiac wall and partially embedded in the targeted vessel, the supply line having a diameter greater than a diameter of the targeted vessel; and seeding angiogenesis due at least in part to the anchored supply line to provide blood flow in the targeted vessel downstream of the blockage.

For purposes of summarizing the disclosure, certain aspects, advantages and novel features have been described. It is to be understood that not necessarily all such advantages may be achieved in accordance with any particular implementation, example, or embodiment. Thus, the disclosed embodiments may be configured to achieve or optimize one advantage or group of advantages as taught herein without necessarily achieving other advantages as may be taught or suggested herein.

### BRIEF DESCRIPTION OF THE DRAWINGS

Various implementations, examples, and embodiments are depicted in the accompanying drawings for illustrative purposes and should in no way be interpreted as limiting the scope of the disclosure. In addition, various features of different disclosed implementations, examples, and embodiments can be combined in various manners, the various combinations being within the scope of this disclosure. Throughout the drawings, reference numbers may be reused to indicate correspondence between reference elements.

FIG. 1 illustrates a perspective view of an example human heart.

FIG. 2 illustrates a cross-sectional view of the example human heart of FIG. 1.

FIGS. 3A, 3B, and 3C illustrate examples of achieving access to coronary arteries via a transfemoral artery catheter that is positioned along the aorta near the aortic arch or the aortic root.

FIG. 4 illustrates an example sealing device to provide hemostasis for catheters advanced out of the aorta.

FIG. 5 illustrates another example sealing device to provide hemostasis, the sealing device configured to be placed prior to advancing the catheter through the puncture.

FIG. 6 illustrates a catheter puncturing out of the aorta near the aortic arch, puncturing and entering the left ventricle, and puncturing the ventricular wall to gain access to a targeted vessel downstream of a blockage.

FIG. 7 illustrates that access to the targeted vessel can be achieved by directly puncturing through the ventricular wall at which point an implant can be placed to keep the puncture open to provide profusion to the targeted vessel.

FIG. 8 illustrates an example of accessing a targeted vessel by gaining access to the pericardial space from the chest of a patient using a pericardiocentesis-like device.

FIG. 9 illustrates a first example mechanism for navigating around a blockage within a targeted vessel that employs a balloon that can be inflated to provide stability for a catheter to puncture out of the targeted vessel.

FIG. 10 illustrates a second example mechanism for navigating around a blockage within a targeted vessel that employs a coil-shaped catheter with an electrocautery tip.

FIG. 11 illustrates an example implant that acts as its own anchor and that directs blood to a targeted vessel using a central lumen and one or more holes.

FIG. 12 illustrates a procedure for accessing an adjacent or nearby artery and implanting a shunt that provides blood flow to a targeted vessel with a blockage.

FIG. 13 illustrates a mechanism for providing stable access to a targeted vessel using a suction-cup mechanism of a catheter.

FIGS. 14A, 14B, and 14C illustrate additional example mechanisms for providing stable access to a targeted vessel using a clip or claw shape that can be part of a catheter.

FIGS. 15A and 15B illustrate another technique to bypass a blockage in a targeted vessel that inserts a transcatheter CABG line directly into the wall of the ventricle near the blocked vessel.

FIGS. 16A, 16B, and 16C illustrate an example mechanism for anchoring a new vessel to a targeted vessel using wires, barbs, and/or hooks.

FIGS. 17A, 17B, 17C, and 17D illustrate an example mechanism wherein anchors are placed prior to puncturing a targeted vessel and act as a guide for puncturing or accessing the targeted vessel.

FIG. 18 illustrates an example mechanism wherein an anchor can be a stent placed in a targeted vessel upstream of a blockage, the stent including a shunt that is anchored through muscle tissue at the targeted vessel.

FIG. 19 illustrates a first visualization example that includes a fiber optic cable in the catheter that can be used to visualize changes in tissue as the vessel is accessed.

FIG. 20 illustrates a second visualization example that includes a catheter can that has a thin-walled section that allows the visualization of angiogram dye on fluoroscopy, allowing the use of flash, similar to the flash on an IV line, to confirm that the vessel has been accessed.

FIG. 21 illustrates a flow chart of an example method for performing CABG procedures.

### DETAILED DESCRIPTION

The headings provided herein are for convenience only and do not necessarily affect the scope or meaning of the claimed subject matter.

### Overview

The following includes a general description of human cardiac anatomy that is relevant to certain features disclosed herein and is included to provide context for certain aspects of the present disclosure. In humans and other vertebrate animals, the heart generally comprises a muscular organ having four pumping chambers, wherein the flow of blood between the pumping chambers is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to associated blood vessels (e.g., pulmonary, aorta, etc.).

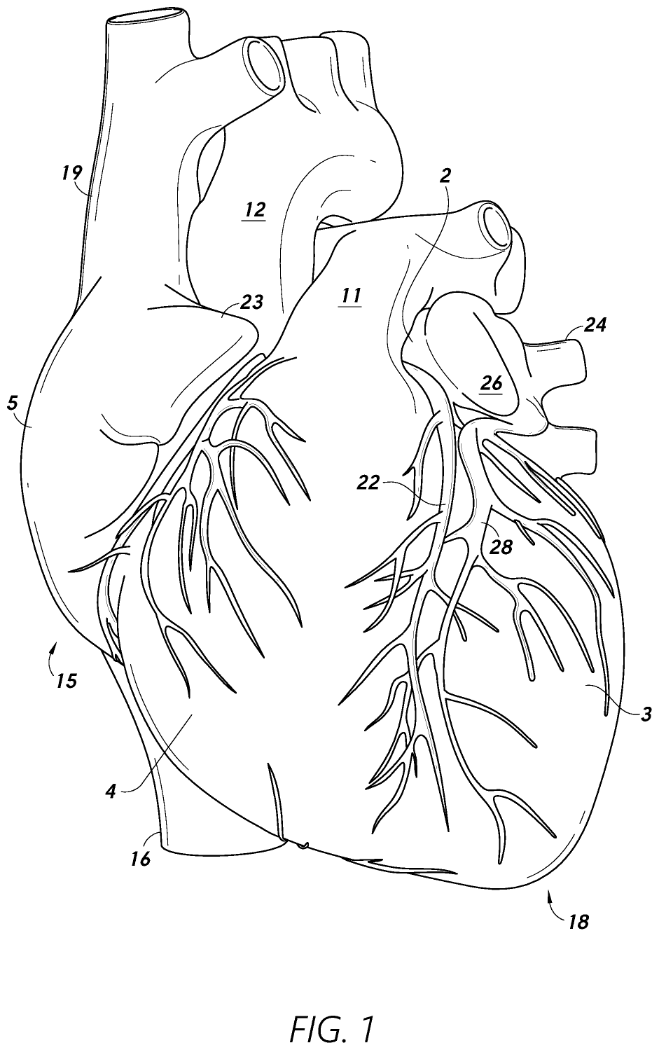

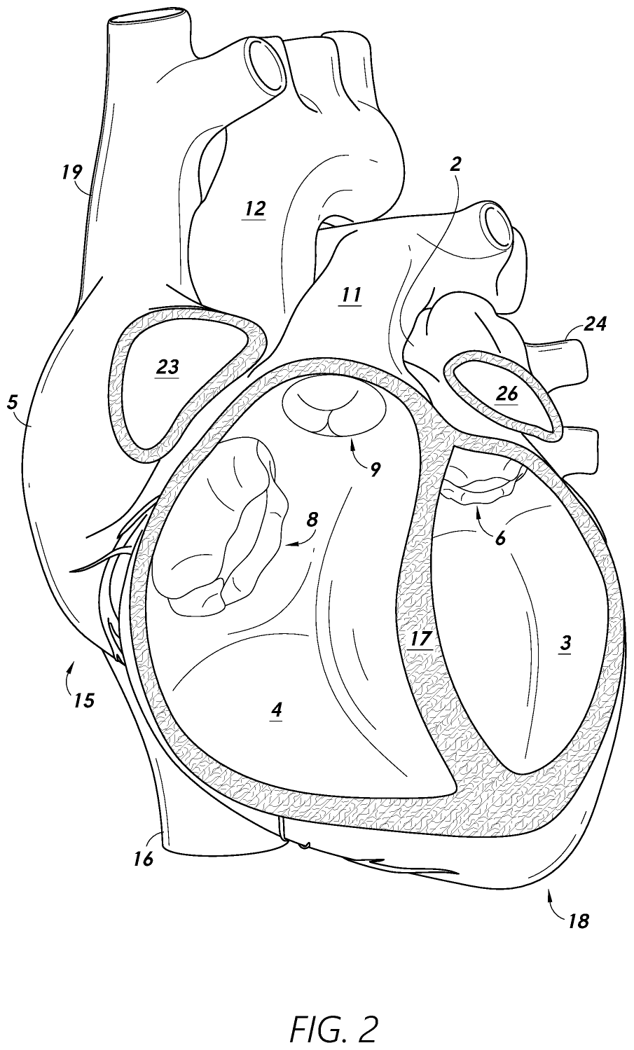

FIGS. 1 and 2 illustrate external perspective and cross-sectional views, respectively, of an example heart having various features/anatomy relevant to certain aspects of the present disclosure. The heart includes four chambers, namely the left ventricle 3, the left atrium 2, the right ventricle 4, and the right atrium 5. A wall of muscle, referred to as the septum, separates the left-side chambers from the right-side chambers. In particular, an atrial septum wall portion (not shown) separates the left atrium 2 from the right atrium 5, whereas a ventricular septum wall portion 17 separates the left ventricle 3 from the right ventricle 4. The inferior tip 18 of the heart is referred to as the apex and is generally located on or near the midclavicular line, in the fifth intercostal space. Attached to the right atrium 5 is the right atrial appendage 23, which generally may comprise a pouch-like extension of the pectinate muscles in the walls of the right atrium 5. Attached to the left atrium 2 is the left atrial appendage 26, which generally may comprise a muscular ear-shaped pouch.

The heart further includes four valves for aiding the circulation of blood therein. The valves of the heart include the tricuspid valve 8, which separates the right atrium 5 from the right ventricle 4. The valves of the heart further include the pulmonary valve 9, which separates the right ventricle 4 from the pulmonary artery 11. The heart further includes the mitral valve 6, which generally has two cusps/leaflets and separates the left atrium 2 from the left ventricle 3. Lastly, the aortic valve (not shown in FIGS. 1 and 2) separates the left ventricle 3 from the aorta 12.

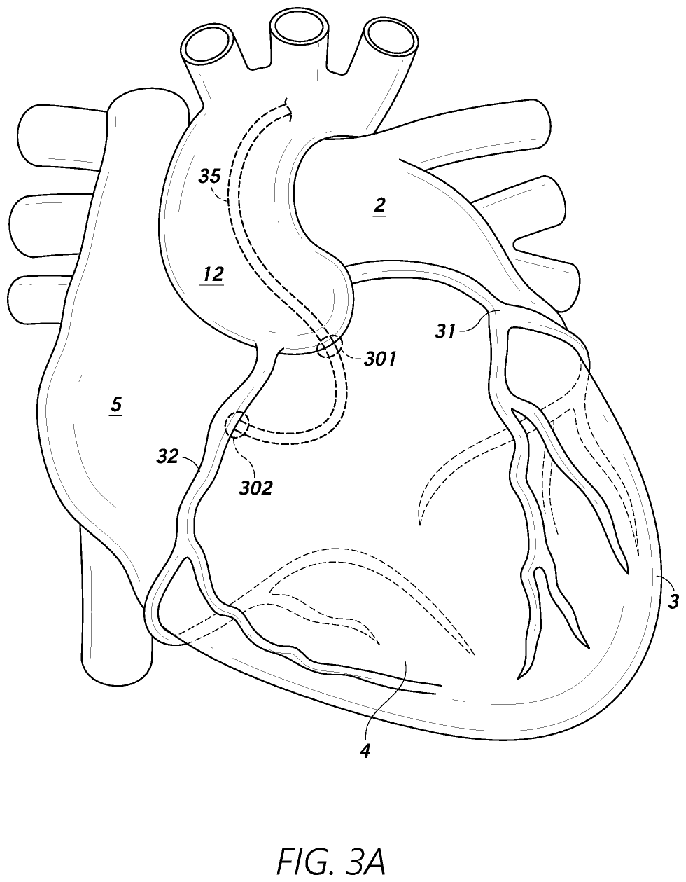

Surrounding the ventricles 3, 4 are a number of coronary arteries 22 that supply oxygenated blood to the heart muscle and a number of coronary veins 28 that return the blood from the heart muscle to the right atrium 5 via the coronary sinus. The coronary arteries 22 include the left coronary artery 31 and the right coronary artery 32 (e.g., see FIGS. 3A-3C). The left coronary artery 31 supplies blood to the left side of the heart muscle (the left ventricle 3 and left atrium 2). The right coronary artery 32 supplies blood to the right side of the heart muscle (the right ventricle 4 and right atrium 5) as well as the sinoatrial and atrioventricular nodes. Both the left and right coronary arteries 31, 32 assist in providing blood to the septum of the heart.

The primary roles of the chambers of the left side of the heart (the left atrium 2 and left ventricle 3) are to act as holding chambers for blood returning from the lungs (not shown) and to act as a pump to transport blood to other areas of the heart. The left atrium 2 receives oxygenated blood from the lungs via the pulmonary veins 24. The oxygenated blood that is collected from the pulmonary veins 24 in the left atrium 2 enters the left ventricle 3 through the mitral valve 6. Deoxygenated blood enters the right atrium 5 through the inferior 16 and superior 19 venae cavae. The right side (the right atrium 5 and right ventricle 4) of the heart then pumps this deoxygenated blood into the pulmonary arteries around the lungs. There, fresh oxygen enters the blood stream, and the blood moves to the left side of the heart via the network of pulmonary veins that ultimately terminate at the left atrium 2, as shown.

Certain physiological conditions or parameters associated with the coronary arteries 31, 32 can impact the health of a patient. For example, coronary artery disorders or disease can reduce the flow of oxygen and nutrients to the heart muscle. This can lead to a heart attack and possibly death. Atherosclerosis (a buildup of plaque in the inner lining of an artery causing it to narrow or become blocked) is the most common cause of heart disease. As described herein, coronary artery bypass procedures provide a way to counteract, correct, or otherwise address these disorders and diseases (e.g., atherosclerosis) by bypassing blockages or obstructions in coronary arteries.

Traditional CABG procedures are performed by accessing the chest cavity and stopping the heart. However, off-pump CABG is becoming more wide-spread in use. Transcatheter CABG procedures are particularly advantageous and thus may become prevalent. A transcatheter CABG procedure can advantageously reduce complications, procedure time, and recovery time as well as providing benefits of being an off-pump, beating heart procedure. Accordingly, disclosed herein are several mechanisms and methods for accessing, implant anchoring, and visualization during a transcatheter CABG procedure.

### Accessing the Targeted Vessel

In a transcatheter CABG procedure, access refers to methods for accessing the blocked coronary artery or arteries (also referred to as “the targeted vessel”) to enable implanting the bypass graft. There are several methods that can be used to access the targeted vessel in CABG procedures, examples of which are provided below.

FIGS. 3A, 3B, and 3C illustrate examples of achieving access to coronary arteries via a transfemoral artery catheter 35 that is advanced from the femoral artery to the heart and positioned along the aorta 12 near the aortic arch or the aortic root. The catheter 35 can be any suitable catheter, including steerable catheters. FIG. 3A illustrates the catheter 35 puncturing into the right atrium 5 to access the right coronary artery 32. The catheter 35 can exit or puncture out of the aortic root at position 301 and can access the right coronary artery 32 at position 302. The position 302 can be upstream or downstream of a blockage of that artery 32. Whether the position 302 is upstream or downstream of the blockage can depend on the methods for bypassing the blockage to be utilized in conjunction with the method of accessing the artery described with respect to FIG. 3A.

FIG. 3B illustrates the catheter 35 puncturing into the pericardial or chest cavity space to navigate a more traditional CABG path to the right or left coronary arteries 31, 32. The catheter 35 can exit or puncture out of the aortic arch at position 303 and can access the left coronary artery 31 at position 304. The position 304 can be upstream or downstream of a blockage of that artery 31. Whether the position 304 is upstream or downstream of the blockage can depend on the methods for bypassing the blockage to be utilized in conjunction with the method of accessing the artery described with respect to FIG. 3B. This approach can also be used to access the right coronary artery 32.

FIG. 3C illustrates the catheter puncturing out of the aorta 12 into the chest cavity space to navigate to the left coronary artery 31 downstream of a blockage 307. The catheter 35 can exit or puncture out of the aortic arch at position 305 and can access the left coronary artery 31 at position 306, which can be downstream of a blockage 307 of that artery. This approach can also be used to access the right coronary artery 32. Although the position 306 is illustrated as being downstream of the blockage 307, the approach described in FIG. 3C can also be used to access the artery 31 upstream of the blockage 307. Whether the position 306 is upstream or downstream of the blockage 307 can depend on the methods for bypassing the blockage to be utilized in conjunction with the method of accessing the artery described with respect to FIG. 3C.

Hemostasis at the site where the aortic root or arch is punctured is advantageously kept while the CABG device is being placed. In some implementations, hemostasis can be achieved with the catheter itself due at least in part to properties of the catheter. In certain implementations, hemostasis can be achieved with the help of a sealing device around the outer diameter of the catheter. The sealing device can be an O-ring, a flap, a Nitinol mesh, a cloth, etc. In some implementations, the sealing device can be a self-expandable, double disk device made from a Nitinol wire mesh, similar to an AMPLATZER™ occluder device.

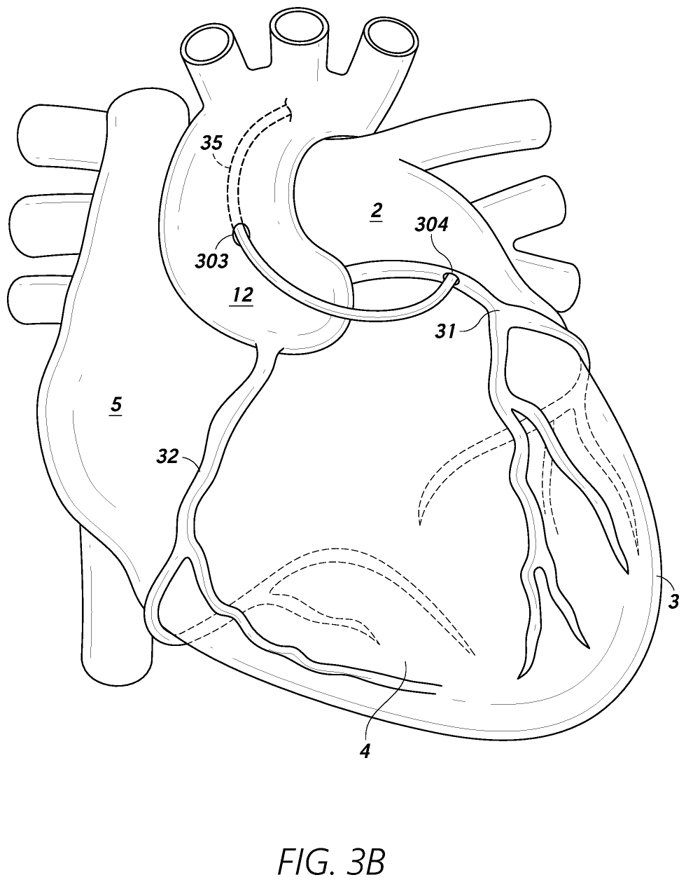

FIG. 4 illustrates an example sealing device 36. The sealing device 36 can be used with any of the methods and devices disclosed herein that puncture the aorta or other vessel or tissue. The sealing device 36 can be configured to remain in place at the aortic puncture 401 while the catheter 35 runs through the puncture 401. In some implementations, advancing the catheter 35 through the puncture 401 advances the sealing device 36 to the puncture 401. In some implementations, the higher pressures within the aorta 12 assist the sealing device 36 to stay in place at the puncture 401 to achieve hemostasis.

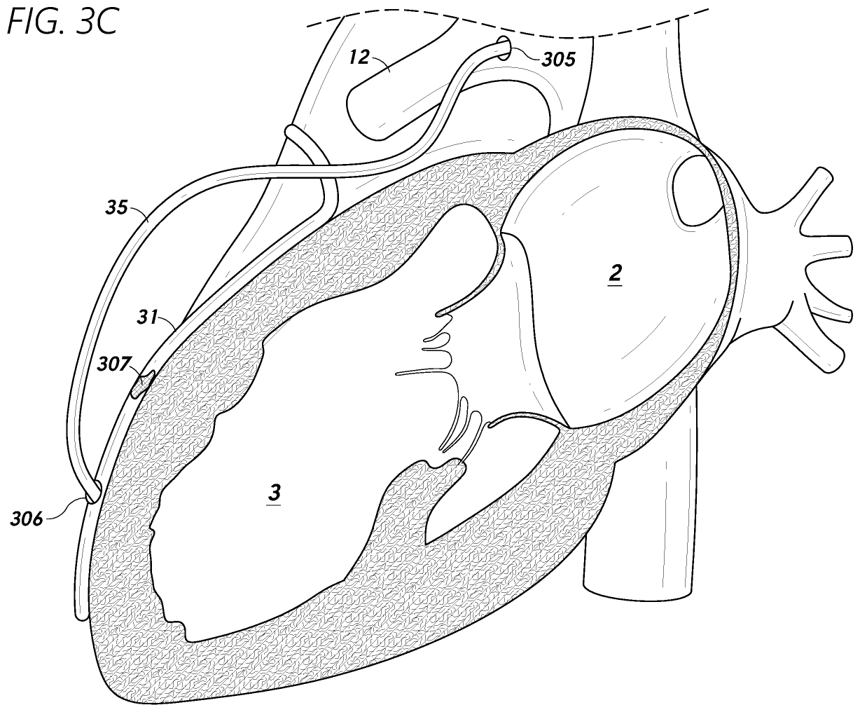

FIG. 5 illustrates another example sealing device 37 that can be placed prior to introducing the catheter 35. The sealing device 37 can be used with any of the methods and devices disclosed herein that puncture the aorta or other vessel or tissue. In some implementations, the sealing device 37 includes a sealed access port through which the catheter 35 navigates. The sealing device 37 thus provides hemostasis while allowing movement of the catheter 35. In certain implementations, the sealing device 37 is configured to grip the puncture 401 to stay in place.

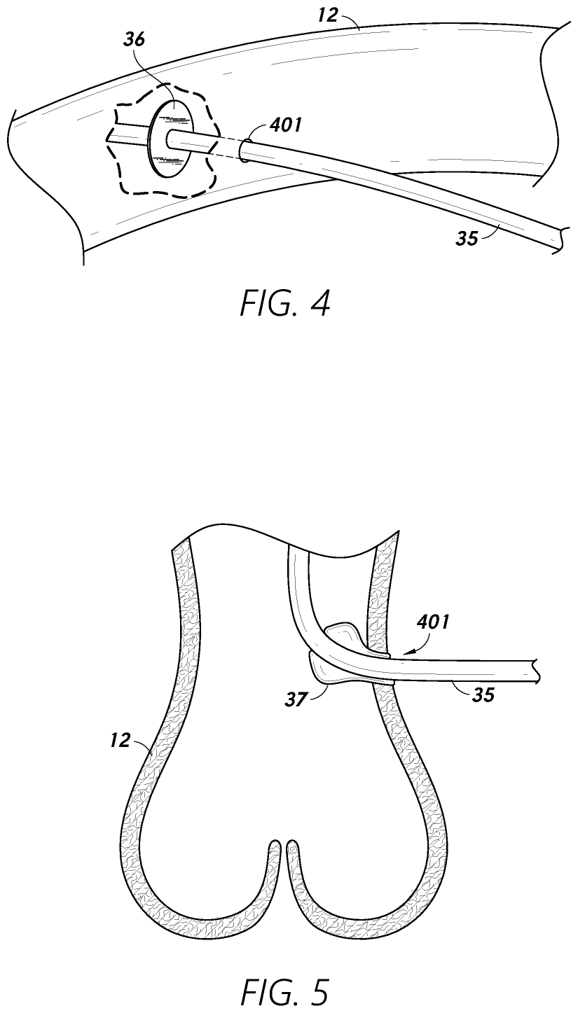

FIG. 6 illustrates the catheter 35 puncturing out of the aorta 12 near the aortic arch at position 601, puncturing and entering the left ventricle 3 at position 602, and puncturing the ventricular wall at position 603 to gain access to the targeted vessel (e.g., the left coronary arteries 31) downstream of a blockage 605. Hemostasis can be achieved using any suitable device or procedure, including those described with respect to FIGS. 4 and 5. Whether access is gained upstream or downstream of the blockage 605 can depend on the methods for bypassing the blockage to be utilized in conjunction with the method of accessing the artery described with respect to FIG. 6.

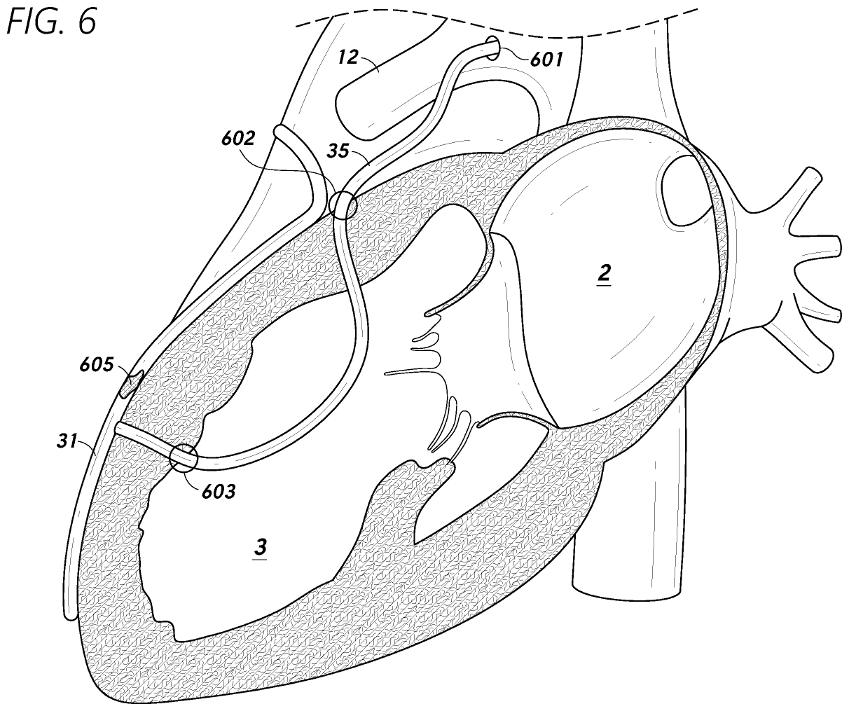

FIG. 7 illustrates that access to the targeted vessel (e.g., the left coronary arteries 31) can be achieved by directly puncturing through the ventricular wall (e.g., the wall of the left ventricle 3). Once accessed is achieved through the ventricular wall, an implant 700 can be placed in the puncture site to ensure the port remains open. In this example, the natural left-side pressures can provide the profusion to the targeted vessel through the implant 700. In some implementations, the implant 700 can be positioned downstream of a blockage 705. Whether access is gained upstream or downstream of the blockage 605 can depend on the methods for bypassing the blockage to be utilized in conjunction with the method of accessing the artery described with respect to FIG. 7.

The implant 700 can be a stent or other similar device. The implant 700 can be configured to have a length sufficient to span the distance from the ventricle (or atrium) to the targeted vessel. In some implementations, the implant 700 can include a one-way valve to maintain the pressure differential between the heart chamber and the targeted vessel. This procedure can also be used to access the left coronary arteries through the left atrial wall.

In some implementations, access is gained to the ventricle through a transeptal approach up the IVC through the mitral valve into the ventricle. Similarly, access to the targeted vessel to place the implant 700 can be gained trans-atrially.

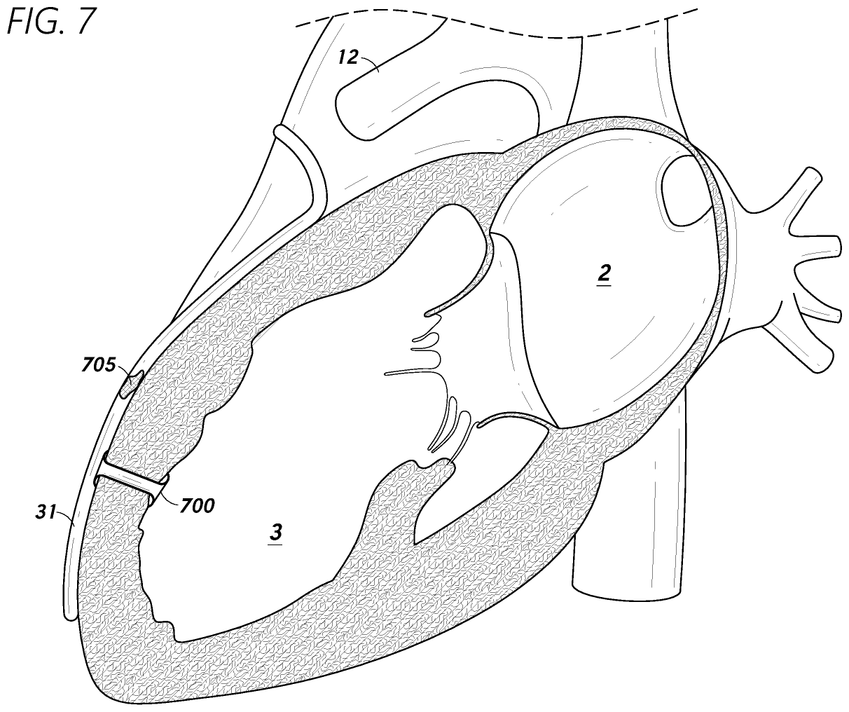

FIG. 8 illustrates an example of accessing a targeted vessel by gaining access to the pericardial space from the chest of a patient 800 using a pericardiocentesis-like device 810. The heart 805 and lungs 802 of the patient 800 are shown merely to provide orientation information. The device 810 can then access the targeted vessel to deliver a CABG implant, to act as a visualization landmark, and/or to deliver part of an implant anchor for a second transcatheter device to engage with. The second transcatheter device can use any of the techniques described herein to target and gain access to the targeted vessel. In such implementations, access with the pericardiocentesis-like device 810 provides a secondary access point.

Access from the chest may be advantageous in situations where it is difficult or impracticable to steer the catheter 35 from the aorta 12 to the targeted vessel. Access from the chest may be advantageous in situations where a first end of a stent is implanted between the aorta and coronary artery using any a transcatheter approach and the other end of the stent is attached using the pericardiocentesis-like device 810 which gains access through the chest. The pericardiocentesis-like device 810 can act as a visualization landmark and/or to provide another visualization catheter to provide direct visualization of the targeted vessel. This may be advantageous to determine a point downstream of a blockage in the targeted vessel. For example, this may be particularly beneficial in situations where there is insufficient dye to adequately provide visualization for guiding the catheter to the targeted vessel.

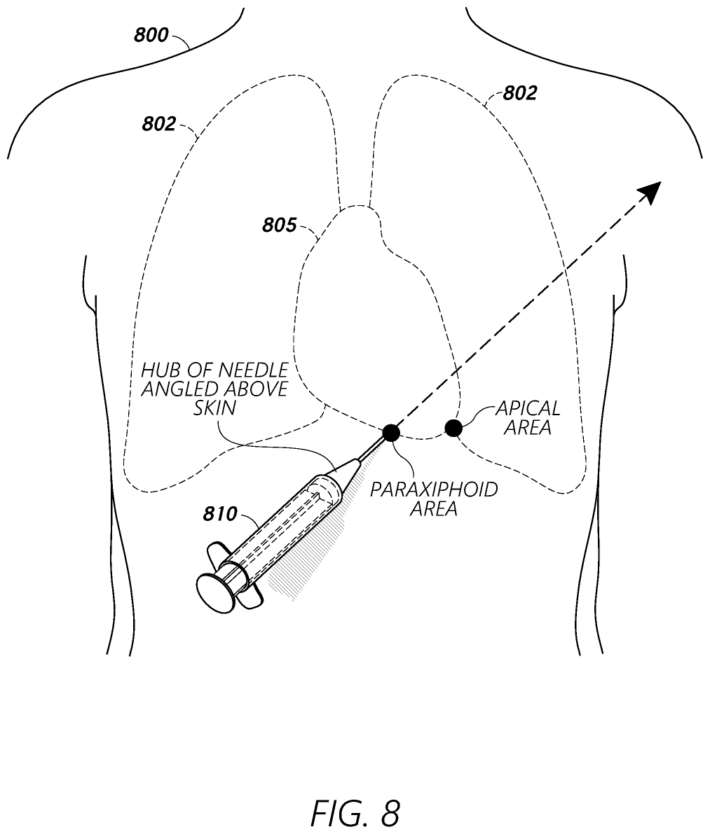

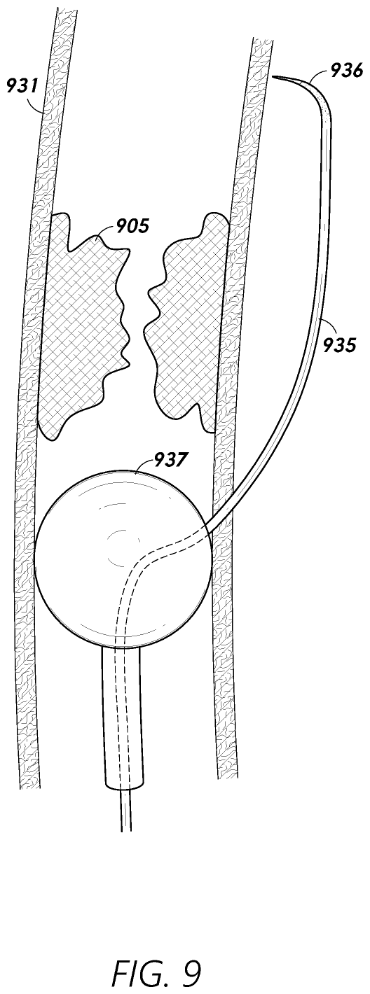

Once access is gained to the targeted vessel using any of the approaches disclosed herein (e.g., the approaches described herein with reference to FIGS. 3A-C and 6-8), one or more of the following mechanisms (e.g., the procedures described herein with reference to FIGS. 9 and 10) can be employed to navigate around a blockage in the targeted vessel. FIG. 9 illustrates a first example mechanism for navigating around a blockage 905 within a targeted vessel 931 that employs a balloon 937 that can be inflated to provide stability for a catheter 935 to puncture out of the targeted vessel 931. The balloon 937 is inflated upstream of the blockage 905. The balloon 937 advantageously provides hemostasis to reduce or prevent bleeding. In some implementations, the balloon 937 can be a directional balloon. The catheter 935 can be an electrocautery catheter with a deflectable tip 936. The catheter 935 can be navigated around the blockage 905 and can re-access the targeted vessel 931 downstream of the blockage 905 using the deflectable tip 936. In some implementations, the catheter 935 can be steerable. In some implementations, the catheter 935 is a directional device, not necessarily deflectable, that has a curve that influences the tip of the catheter 935 to re-enter the targeted vessel.

The catheter 935 establishes a path for implant delivery. For example, the catheter 935 can include a guidewire that provides a rail for a bypass shunt. In some implementations, the guidewire of the catheter 935 can be shape-set.

FIG. 10 illustrates a second example mechanism for navigating around a blockage 1005 within a targeted vessel 1031 that employs a coil-shaped catheter 1035 with an electrocautery tip 1036. The catheter 1035 can be fed out of the targeted vessel 1031 near the blockage 1005 and the coil shape can provide stability following the targeted vessel 1031 past the blockage 1005 where it can re-enter the targeted vessel 1031. The catheter 1035 can exit the targeted vessel 1031 at position 1001 upstream of the blockage 1005 and can re-enter the targeted vessel 1031 at position 1002 downstream of the blockage 1005. The catheter 1035 can leave a guidewire path for implant delivery, similar to what is described herein with respect to FIG. 9.

In some implementations, there is a directional bias in the coiled shape of the catheter 1035. In such implementations, advancing the catheter 1035 causes it to advance out of the targeted vessel 1031. In some implementations, the catheter 1035 includes a directional port at the exit, like the balloon catheter 935 described herein with respect to FIG. 9. The coiled shape of the catheter 1035 is configured to follow the targeted vessel 1031 after exiting the targeted vessel 1031 at the exit point 1001. In some implementations, the tip 1035 is deflectable to control the re-entry point 1002. In some implementations, tension can be used to cinch down the catheter 1035 on the targeted vessel 1031.

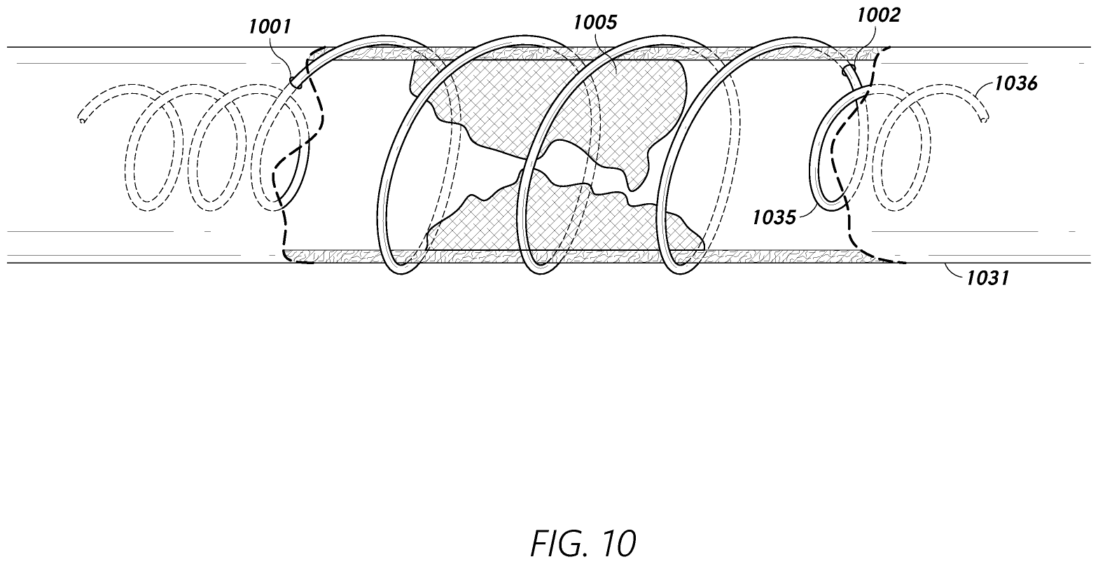

Once access is gained to the targeted vessel using any of the approaches disclosed herein (e.g., the approaches described herein with reference to FIGS. 3A-3C and 6-8), a suitable implant can be implanted to provide profusion. FIG. 11 illustrates an example implant 1135 that acts as its own anchor and that directs blood to a targeted vessel using a central lumen 1139 and one or more holes or perforations 1137. The implant 1135 is coil-shaped with a central lumen 1139 and acts as its own anchor. For example, the implant 1135 can anchor through a ventricular wall 1103 into a targeted vessel. The central lumen 1139 provides a fluidic path for blood from the ventricle to be directed into the targeted vessel. The circle inset of FIG. 11 shows a tip portion of the implant 1135 in cross-section to better illustrate the central lumen 1139. The central lumen 1139 extends the length of the implant 1135. The implant 1135 can be a coil-shaped shunt. In some implementations, the implant 1135 is delivered inside the ventricle and is implanted downstream of a blockage in the targeted vessel. In some implementations, the implant 1135 includes a plurality of holes 1137 to facilitate the transfer of blood from the heart chamber to the targeted vessel. Placement of the implant 1135 to provide profusion can be similar to the placement of the implant 700 described herein with reference to FIG. 7. For example, placement of the implant 1135 can be through a wall of the left ventricle 3 into the left coronary arteries 31 downstream of the blockage 705, as illustrated in FIG. 7. It should be understood that a similar placement of the implant 1135 can be made into the right coronary arteries 32 from the right atrium or ventricle.

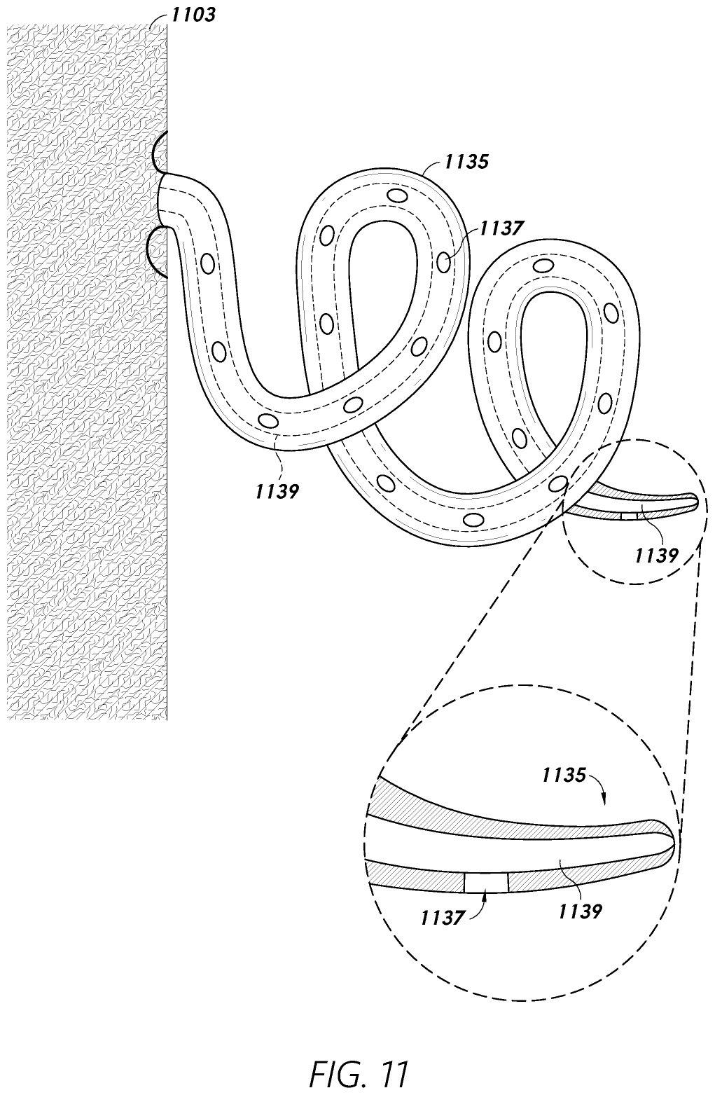

Another approach for bypassing a blockage includes accessing a first coronary artery that is functioning normally and shunting from the functioning coronary artery to a blocked artery downstream of a blockage. Accessing the first coronary artery can be achieved using any of the approaches disclosed herein (e.g., the approaches described herein with reference to FIGS. 3A-3C and 6-8). FIG. 12 illustrates a procedure for accessing an adjacent or nearby artery 31 and implanting a shunt 1200 that provides blood flow to a targeted vessel 32 downstream of a blockage 1205. In some implementations, this can be done from an artery that is on the opposite side of the heart. Once accessed, a shunt can be created between the arteries using any suitable mechanism and/or device. This may be facilitated through the use of a steerable catheter.

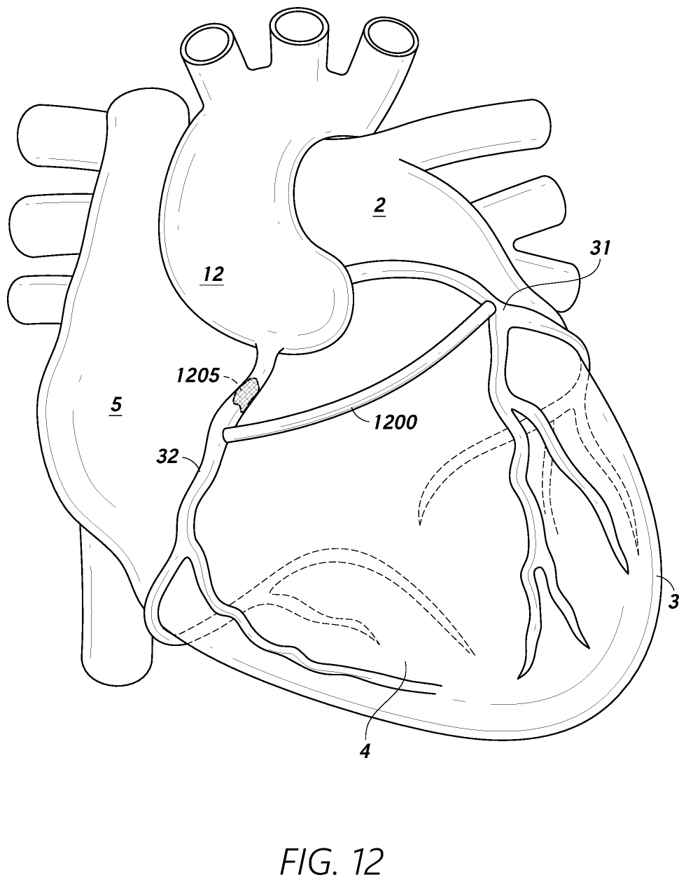

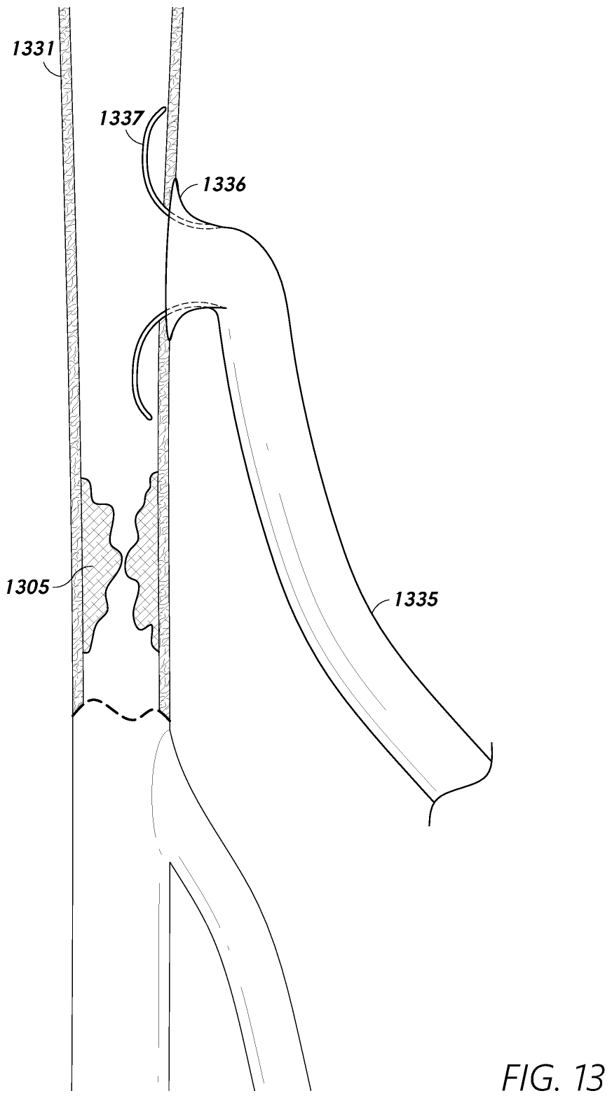

Once the blocked artery is accessed past the blockage using any suitable mechanism such as those described herein (e.g., the mechanisms described herein with reference to FIGS. 3A-3C, 6-10, and 12), it is beneficial to enable stable access to the targeted vessel. Example mechanisms for providing stable access to the targeted vessel are provided in FIGS. 13-14C.

FIG. 13 illustrates a mechanism for providing stable access to a targeted vessel 1331 using a suction-cup mechanism 1336 of a catheter 1335. The suction-cup mechanism 1336 provides a way of sealing and positioning the tip of the locating catheter 1335. The suction-cup mechanism 1336 is configured to provide a seal that enables puncturing into the targeted vessel 1331 to enable deployment of a shunt. The catheter 1335 includes anchor flanges 1337 to anchor the catheter 1335 to the targeted vessel 1331 downstream and/or upstream of the blockage 1305. The anchor flanges 1337 assist in anchoring the catheter 1335 and in creating a temporary seal. An implant (e.g., a shunt) can be deployed through the catheter 1335. The catheter 1335 enables access to the targeted vessel from outside of the vessel while providing hemostasis. In some implementations, the catheter 1335 includes a deflectable tip to position the suction-cup mechanism 1336. In some implementations, the suction-cup mechanism 1336 includes a flared conical structure that provides contact on the targeted vessel 1331. In some implementations, the structural design of the suction-cup mechanism 1336 is configured to provide a suction-force on the targeted vessel 1331 to aid in retaining contact of the catheter with the targeted vessel 1331.

FIGS. 14A, 14B, and 14C illustrate additional example mechanisms for providing stable access to a targeted vessel using a clip or claw shape that can be part of a catheter. The clip or claw shape is configured to grab the targeted vessel. In some implementations, this clip or claw shape can also act as part of the implant. The clip 1400 (FIGS. 14A and 14B) or claw 1410 (FIG. 14C) can be configured to stabilize the targeted vessel for puncturing. The clip 1400 (FIGS. 14A and 14B) or claw 1410 (FIG. 14C) is configured to grip the targeted vessel from outside the vessel. This may be particularly advantageous when accessing the targeted vessel from outside of the vessel. As illustrated in FIGS. 14A and 14B, the clip 1400 includes a sliding portion that acts to open and close the clip portion of the clip 1400. Similarly, as illustrated in FIG. 14C, the claw 1410 includes a mechanism for opening and closing the claw on the vessel. In some implementations, the claw 1410 acts as an anchor within the targeted vessel. The clip 1400 (FIGS. 14A and 14B) or claw 1410 (FIG. 14C) can be used to provide stable access to the targeted vessel similar to the way the suction-cup mechanism 1336 is used to provide stable access to the targeted vessel 1331, as illustrated in FIG. 13.

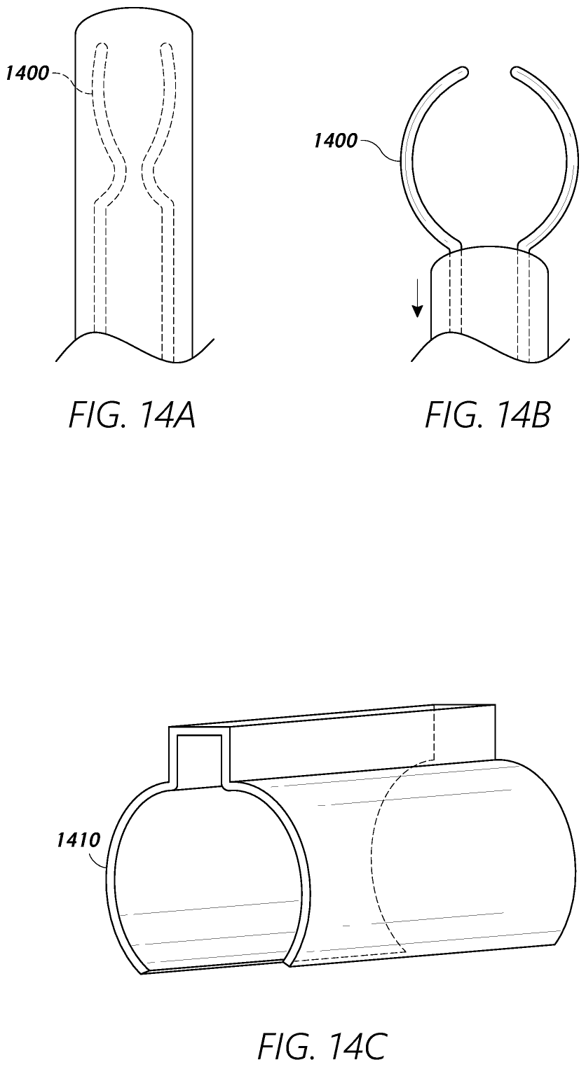

FIGS. 15A and 15B illustrate another technique to bypass a blockage 1505 in a targeted vessel 1531 that inserts a transcatheter CABG line 1500 directly into the wall of the ventricle 1507 near the blocked vessel 1531. Over a period of time, angiogenesis can occur. This allows the heart to be oxygenated past the blockage 1505. This result can also be achieved more directly by positioning an oversized CABG line 1500 partially in the vessel 1531 and partially in the ventricular wall 1507, as illustrated in FIG. 15A. Using this technique, a foam or skirt sealing mechanism 1506 can be used, as illustrated in FIG. 15B. The supply line 1500 can have one end implanted at the targeted vessel 1531 and the other end implanted at the aorta, in the targeted vessel upstream of the blockage 1505, at another coronary artery, or at any other suitable location.

In some implementations, the supply line 1500 can be oversized (e.g., larger than the targeted vessel 1531). In some implementations, the supply line is about 8 mm in diameter (compared to a targeted vessel 1531 with a diameter of about 4 mm). Thus, the supply line 1500 can have a diameter between about 6 mm and about 12 mm or a diameter that is between about 1.5 times and about 4 times the diameter of the targeted vessel 1531. The supply line 1500 is partially implanted into the vessel 1531 and partially outside the vessel due at least in part to its diameter being larger than the diameter of the targeted vessel 1531.

This mechanism may be particularly advantageous when visualization of the blockage is difficult or impracticable. This mechanism is also advantageous in that it adds new vasculature. This mechanism is also advantageous due at least in part to it being used when targeted vessels are relatively small rather than attempting to match the small diameter of a targeted vessel with an implant. This mechanism can be used in combination with any of the mechanisms for accessing a targeted vessel described herein, such as those described herein with reference to FIGS. 3A-3C, 6-10, and 12.

### Implant Anchoring

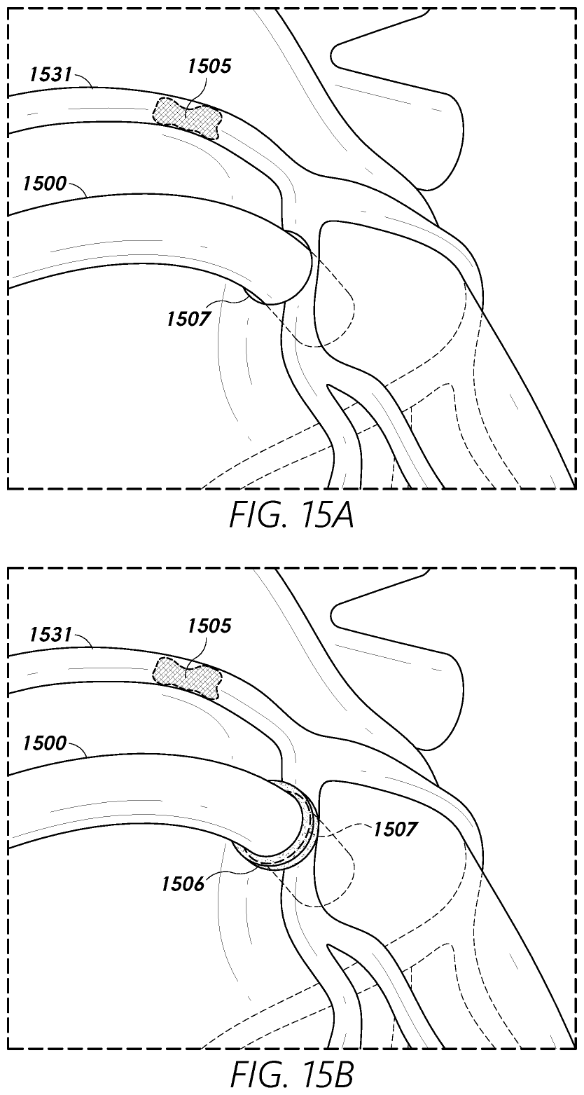

In addition to accessing to the blocked vessel using any of the techniques or mechanisms described herein (e.g., as described in FIGS. 3A-3C, 6-10, and 12), an implant or graft can be anchored to enable bypassing of the blockage. FIGS. 16A, 16B, and 16C illustrate an example mechanism for anchoring a new vessel 1600 to a targeted vessel 1631 using wires, barbs, and/or hooks 1602. The new vessel 1600 can be delivered using a steerable catheter that includes the new vessel material 1600. The new vessel 1600 includes anchors 1602 that are anchored to the targeted vessel 1631 downstream of the blockage 1605. The catheter steers to the targeted vessel 1631 (see FIG. 16A) and punctures the targeted vessel 1631 (see FIG. 16B). Once punctured, the anchors 1602 are deployed to anchor the new vessel 1600 to the targeted vessel 1631 (see FIG. 16C). This establishes a path into the targeted vessel 1631 past the blockage 1605. The anchors 1602 can be Nitinol barbs, wires, hooks, or the like. In some implementations, the new vessel 1600 can be the shunt 1200 of FIG. 12 or the vessel 1500 of FIGS. 15A and 15B.

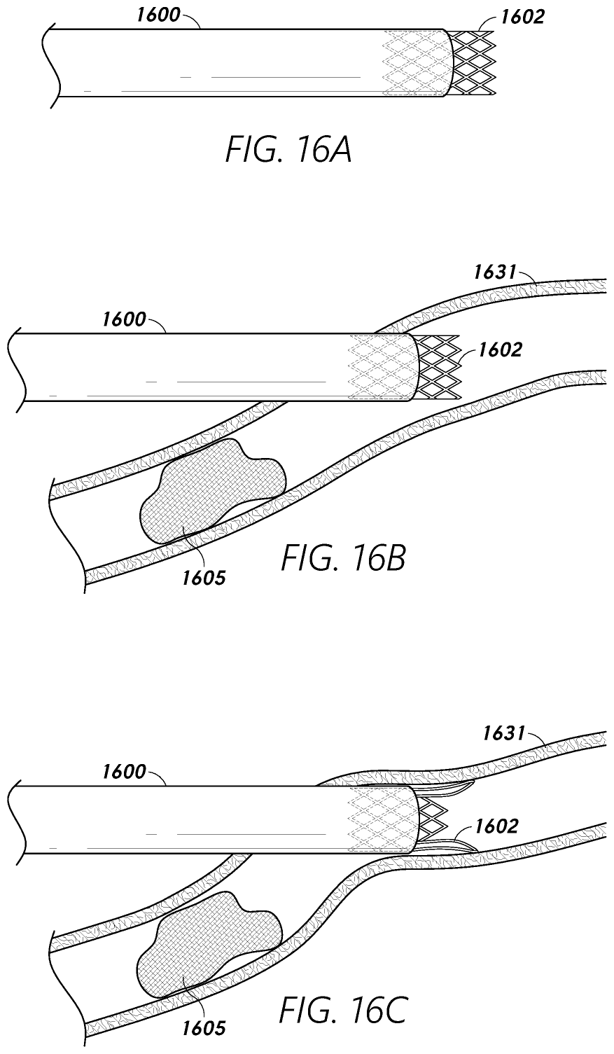

FIGS. 17A, 17B, 17C, and 17D illustrate an example mechanism wherein anchors 1702 are placed prior to puncturing a targeted vessel 1731 and act as a guide for puncturing or accessing the targeted vessel 1731. The new vessel 1700 can be delivered using a steerable catheter that includes the new vessel material 1700. The new vessel 1700 includes anchors 1702 that are anchored to the targeted vessel 1731 downstream of the blockage prior to puncturing the targeted vessel 1731. The catheter steers to the targeted vessel 1731 (see FIG. 17A), inserts the anchors 1702 into the targeted vessel 1731 (see FIG. 17B), and then deploys the anchors 1702 to anchor the new vessel 1700 to the targeted vessel 1700 (see FIG. 17C). Once anchored, the catheter punctures the targeted vessel 1731 to start the flow past the blockage (see FIG. 17D). This establishes a path into the targeted vessel 1731 past the blockage. The anchors 1702 can be Nitinol barbs, wires, hooks, or the like. In some implementations, the new vessel 1700 can be the shunt 1200 of FIG. 12 or the vessel 1500 of FIGS. 15A and 15B.

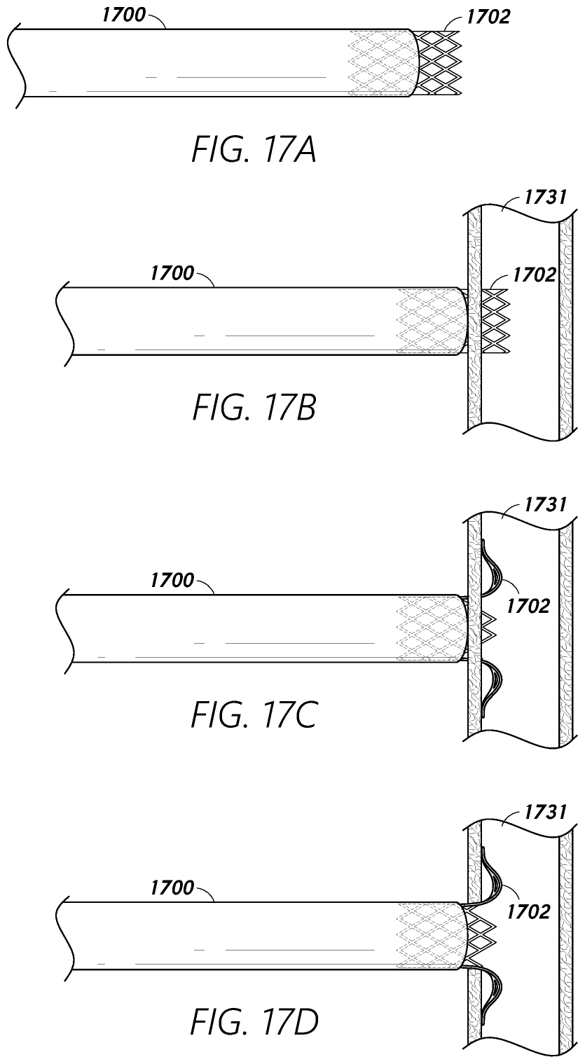

FIG. 18 illustrates an example mechanism wherein an implant can be a stent 1802 placed in a targeted vessel 1831 upstream of a blockage 1805. The stent 1802 can be attached to a CABG line (e.g., shunt 1803) and anchored downstream of the blockage 1805 using a helical anchor 1804. The anchor 1804 can be, for example, an anchor similar to anchors used with CARDIOBAND™ or ROOT™ systems. In some implementations, the stent 1802 can be adjustable to modulate flow. In such implementations, the adjustable stent 1802 can be expanded as needed. In some implementations, the anchor 1804 can be anchored through muscle tissue at or near the targeted vessel 1831. The stent 1802 can be a covered stent that is implanted upstream of the blockage 1805. The shunt 1803 is implanted so that it bypasses the blockage 1805. The shunt 1803 is anchored downstream of the blockage 1805 to provide a bypass path around the blockage 1805. The anchor 1804 can be a helical anchor designed to penetrate and to anchor to muscle tissue. The anchor 1804 can be anchored through muscle tissue that is around the targeted vessel 1831. Access can be gained upstream of the blockage 1805 using any suitable access procedure or mechanism, such as those described herein with reference to FIGS. 3A-3C and 6-8.

### Visualization

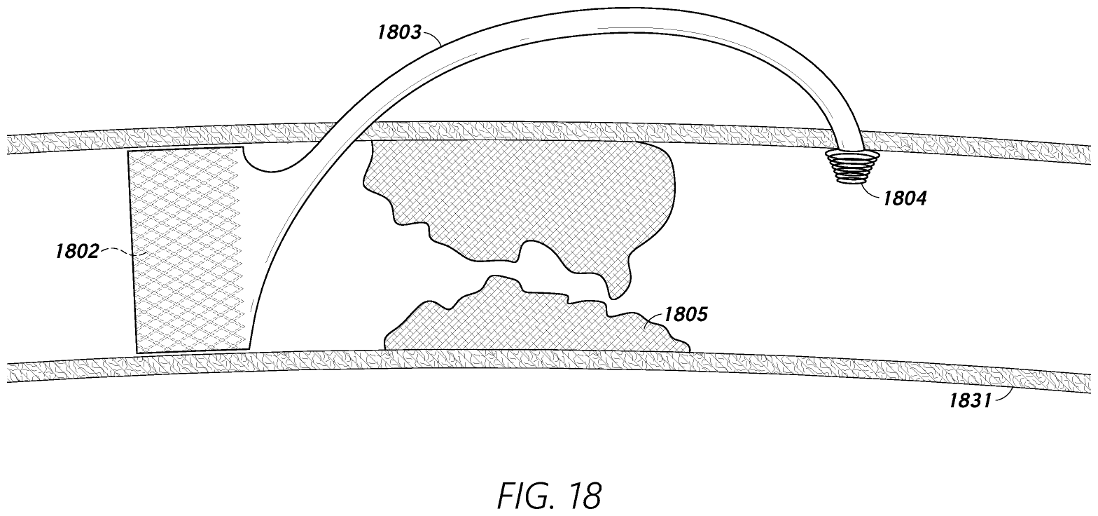

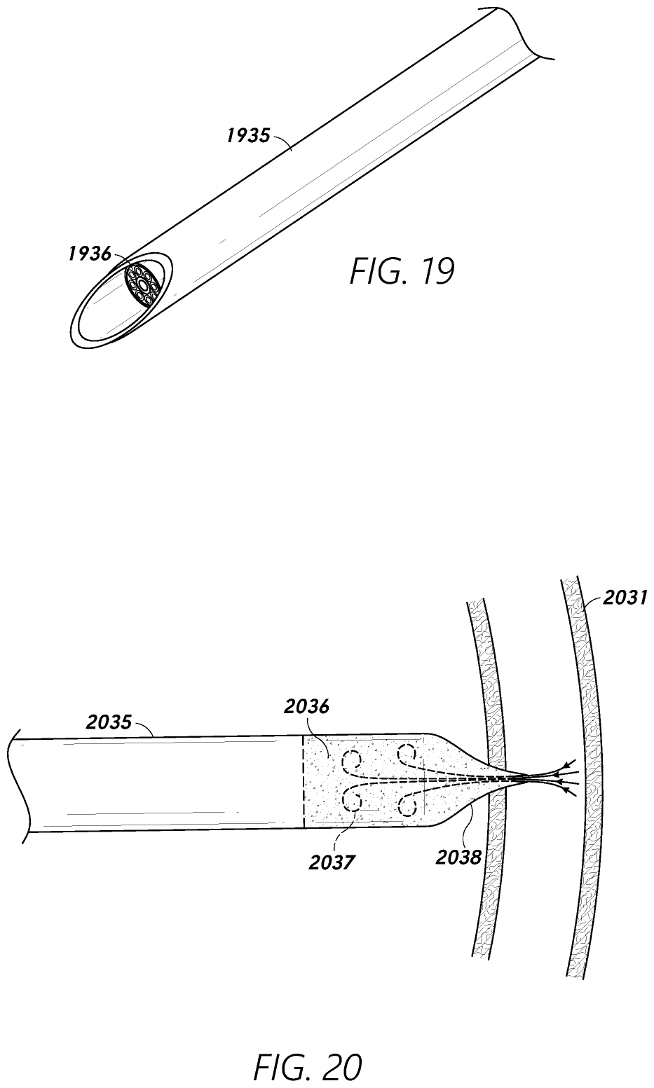

Several mechanisms can be used to visualize the target anchoring area of the vessel past the blockage. For example, an intravascular ultrasound (IVUS) catheter can be used to detect flow in the vessel and a central lumen in the IVUS catheter can house the puncture needle and CABG implant. As another example, an inflated balloon with a direct visual camera can be used to find the vessel. The mechanisms used to visualize the target vessel (e.g., the mechanisms described with reference to FIGS. 19 and 20) can be used in combination with any of the mechanisms or procedures described herein with reference to FIGS. 3A-18.

FIG. 19 illustrates a visualization example that includes a fiber optic cable 1936 in the catheter 1935 that can be used to visualize changes in tissue as the vessel is accessed. In this way, the catheter 1935 can provide direct visualization of the targeted vessel and potential anchor and/or puncture site.

FIG. 20 illustrates a second visualization example that includes a catheter 2035 that has a thin-walled section 2036 that allows the visualization of angiogram dye 2037 on fluoroscopy. This allows the use of flash, similar to the flash on an IV line, to confirm that the targeted vessel 2031 has been accessed. For example, the catheter 2035 includes a tip 2038 that punctures the targeted vessel 2031. Upon puncturing the targeted vessel 2031, the dye will mix and be visible through proper imaging techniques. This flash confirms that the targeted vessel 2031 has been punctured.

In the implementations described herein, an EP-style force gage catheter can be used to gage the hardness of the vessel, navigating past the calcified areas to the soft tissue for the puncture site. This can be used in addition to or as an alternative to the described visualization mechanisms.

### Example CABG Methods

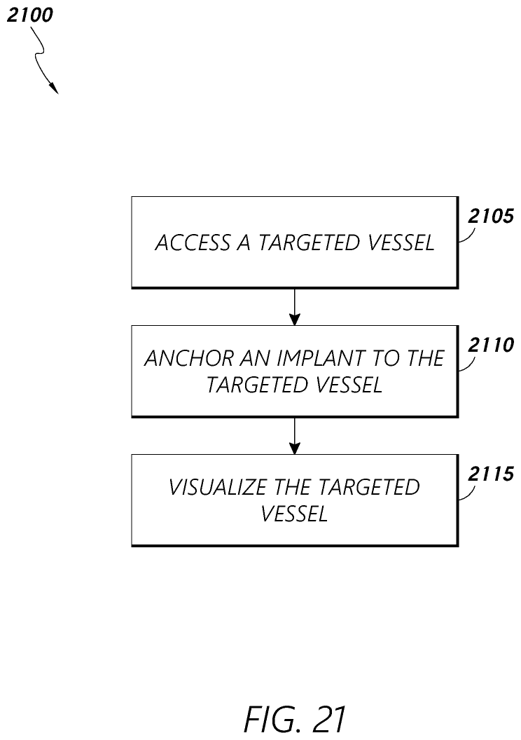

FIG. 21 illustrates a flow chart of an example method 2100 for performing CABG procedures. At block 2105, a targeted vessel is accessed. This can be achieved using any suitable mechanism, including the methods and devices described herein with respect to FIGS. 3A-15B. The targeted vessel can be a coronary artery with a blockage that is targeted for bypassing. At block 2110, an implant is anchored to the targeted vessel. This can be achieved using any suitable mechanism, including the methods and devices described herein with respect to FIGS. 16-18. The implant can be a shunt, a new vessel, or other implant configured to improve or increase the flow of blood in the targeted vessel downstream of a blockage. At block 2115, the targeted vessel is visualized. This can be achieved using any suitable mechanism, including the methods and devices described herein with respect to FIGS. 19 and 20. Visualization can occur concurrently with block 2105 and/or block 2110.

### Additional Features and Embodiments

The following includes additional description of examples. The examples are intended to illustrate examples of combinations of elements and are not intended to limit the scope of any particular example, implementation, or embodiment disclosed herein.

Example 1: A method for performing cardiac artery bypass grafting. The method includes navigating a transfemoral artery catheter to an aorta of a patient; puncturing through the aorta with the transfemoral artery catheter; navigating the transfemoral artery catheter to a targeted vessel with a blockage; and implanting a shunt to bypass the blockage.

Example 2: The method of any example herein, in particular example 1, wherein puncturing through the aorta comprises puncturing into a right atrium to access a right coronary artery.

Example 3: The method of any example herein, in particular example 1, wherein puncturing through the aorta comprises puncturing into a pericardial space to access a right coronary artery or a left coronary artery.

Example 4: The method of any example herein, in particular example 1, wherein puncturing through the aorta comprises puncturing an aortic arch of the aorta.

Example 5: The method of any example herein, in particular example 1, wherein puncturing through the aorta comprises puncturing an aortic root of the aorta.

Example 6: The method of any example herein, in particular example 1, further comprising puncturing through a ventricular wall to access the targeted vessel.

Example 7: The method of any example herein, in particular example 6, further comprising puncturing into a ventricle from a pericardial space prior to puncturing through the ventricular wall to access the targeted vessel.

Example 8: The method of any example herein, in particular examples 1-7, further comprising advancing a sealing device to a puncture location comprising a location where the transfemoral artery catheter punctures through the aorta.

Example 9: The method of any example herein, in particular example 8, wherein the sealing device is secured to a portion of the transfemoral artery catheter so that advancing the transfemoral artery catheter causes the sealing device to contact material around the puncture location to achieve hemostasis.

Example 10: The method of any example herein, in particular example 8, wherein the sealing device is attached to the aorta prior to introducing the transfemoral artery catheter through the aorta.

Example 11: A method for performing cardiac artery bypass grafting. The method includes navigating a catheter to a ventricle of a patient; puncturing through a ventricular wall into a targeted vessel with a blockage; and implanting a shunt between the ventricle and the targeted vessel such that the shunt is downstream of the blockage and the shunt enables passage of blood from the ventricle to the targeted vessel.

Example 12: The method of any example herein, in particular example 11, wherein the shunt comprises a coil-shaped anchor with a central lumen.

Example 13: The method of any example herein, in particular example 12, wherein the shunt further comprises a plurality of perforations in the shunt to facilitate transfer of blood.

Example 14: A method for performing cardiac artery bypass grafting. The method includes navigating a catheter to a first coronary artery of a patient; puncturing through the first coronary artery of the patient; navigating the catheter to a second coronary artery of the patient, the second coronary artery including a blockage; and implanting a shunt from the first coronary artery of the patient to the second coronary artery of the patient, the shunt being anchored to the second coronary artery of the patient downstream of the blockage.

Example 15: A method for performing cardiac artery bypass grafting. The method includes navigating a catheter within a targeted vessel of a patient, the targeted vessel including a blockage, the catheter navigated to a position upstream of the blockage; puncturing through the targeted vessel with the catheter; navigating the catheter outside the targeted vessel to a point downstream of the blockage; and re-entering the targeted vessel with the catheter downstream of the blockage.

Example 16: The method of any example herein, in particular example 15, wherein the catheter comprises a coiled configuration to wrap around the targeted vessel.

Example 17: The method of any example herein, in particular examples 15-16, wherein the catheter comprises a deflectable tip.

Example 18: The method of any example herein, in particular examples 15-17, wherein the catheter comprises an electrocautery tip.

Example 19: The method of any example herein, in particular examples 15-18, further comprising inflating a balloon to stabilize the catheter for puncturing through the targeted vessel.

Example 20: A method for performing cardiac artery bypass grafting. The method includes navigating a catheter to a targeted vessel of a patient, the targeted vessel including a blockage, the catheter navigated to a position downstream of the blockage; anchoring an implant to the targeted vessel, the implant including anchors at a distal end of the implant configured to secure to the targeted vessel; and creating a passageway from the implant to the targeted vessel to enable blood flow in the targeted vessel downstream of the blockage.

Example 21: The method of any example herein, in particular example 20, wherein the passageway is created after anchoring the implant to the targeted vessel.

Example 22: The method of any example herein, in particular example 20, wherein the passageway is created prior to anchoring the implant to the targeted vessel.

Example 23: The method of any example herein, in particular examples 20-22, wherein creating the passageway comprises implanting a stent within the targeted vessel upstream of the blockage and anchoring a distal end of a shunt downstream of the blockage, the shunt providing the passageway between the stent and the targeted vessel at the distal end of the shunt.

Example 24: The method of any example herein, in particular example 23, wherein the distal end of the shunt includes a helical anchored that is anchored into muscle tissue at the targeted vessel.

Example 25: The method of any example herein, in particular examples 20-24, further comprising directly visualizing the targeted vessel with a fiber optic cable that is part of the catheter.

Example 26: The method of any example herein, in particular examples 20-24, further comprising visualizing angiogram dye on fluoroscopy responsive to a tip of the catheter being inserted into the targeted vessel.

Example 27: A method for performing cardiac artery bypass grafting. The method includes navigating a catheter to a targeted vessel of a patient, the targeted vessel including a blockage, the catheter navigated to a position downstream of the blockage; anchoring a supply line that is partially embedded in a cardiac wall and partially embedded in the targeted vessel, the supply line having a diameter greater than a diameter of the targeted vessel; and seeding angiogenesis due at least in part to the anchored supply line to provide blood flow in the targeted vessel downstream of the blockage.

Although certain preferred embodiments and examples are disclosed herein, inventive subject matter extends beyond the specifically disclosed examples, implementations, and embodiments to other alternatives and/or uses, and to modifications and equivalents thereof. Thus, the scope of the claims that may arise herefrom is not limited by any of the particular embodiments described herein. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The above description of embodiments of the disclosure is not intended to be exhaustive or to limit the disclosure to the precise form disclosed above. While specific implementations, embodiments, and examples are described herein for illustrative purposes, various equivalent modifications are possible within the scope of the disclosure, as those skilled in the relevant art will recognize.

Certain terms of location are used herein with respect to the various disclosed embodiments. Although certain spatially relative terms, such as “outer,” “inner,” “upper,” “lower,” “below,” “above,” “vertical,” “horizontal,” “top,” “bottom,” and similar terms are used herein to describe a spatial relationship of one device/element or anatomical structure relative to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described may be performed in any suitable or desirable temporal order. Furthermore, any of the illustrated and/or described operations or steps may be omitted from any given method or process, and the illustrated/described methods and processes may include additional operations or steps not explicitly illustrated or described.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled,” as generally used herein, refers to two or more elements that may be physically, mechanically, and/or electrically connected or otherwise associated, whether directly or indirectly (e.g., via one or more intermediate elements, components, and/or devices. Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number may also include the plural or singular number, respectively.

The word “or” in reference to a list of two or more items, that word covers all of the following interpretations of the word: any of the items in the list, all of the items in the list, and any combination of the items in the list. Furthermore, as used herein, the term “and/or” used between elements (e.g., between the last two of a list of elements) means any one or more of the referenced/related elements. For example, the phrase “A, B, and/or C” means “A,” “B,” “C,” “A and B,” “A and C,” “B and C,” or “A, B, and C.”

As may be used herein, the terms “substantially” and “approximately” provides an industry-accepted tolerance for its corresponding term and/or relativity between items. For some industries, an industry-accepted tolerance is less than one percent, while for other industries, the industry-accepted tolerance may be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than approximately +/−1%). Some relativity between items may range from a difference of less than a percentage level to a few percent. Other relativity between items may range from a difference of a few percent to magnitude of differences.

What is claimed is:

1. A method for performing cardiac artery bypass grafting, the method comprising:
navigating a transfemoral artery catheter to an aorta of a patient;
puncturing through the aorta with the transfemoral artery catheter;
navigating the transfemoral artery catheter to a targeted vessel with a blockage; and
implanting a shunt to bypass the blockage.

2. The method of claim 1, wherein puncturing through the aorta comprises puncturing into a right atrium to access a right coronary artery.

3. The method of claim 1, wherein puncturing through the aorta comprises puncturing into a pericardial space to access a right coronary artery or a left coronary artery.

4. The method of claim 1, wherein puncturing through the aorta comprises puncturing an aortic arch of the aorta.

5. The method of claim 1, wherein puncturing through the aorta comprises puncturing an aortic root of the aorta.

6. The method of claim 1 further comprising puncturing through a ventricular wall to access the targeted vessel.

7. The method of claim 6 further comprising puncturing into a ventricle from a pericardial space prior to puncturing through the ventricular wall to access the targeted vessel.

8. The method of claim 1 further comprising advancing a sealing device to a puncture location comprising a location where the transfemoral artery catheter punctures through the aorta.

9. The method of claim 8, wherein the sealing device is secured to a portion of the transfemoral artery catheter so that advancing the transfemoral artery catheter causes the sealing device to contact material around the puncture location to achieve hemostasis.

10. The method of claim 8, wherein the sealing device is attached to the aorta prior to introducing the transfemoral artery catheter through the aorta.

11. A method for performing cardiac artery bypass grafting, the method comprising:
navigating a catheter to a ventricle of a patient;
puncturing through a ventricular wall into a targeted vessel with a blockage; and
implanting a shunt between the ventricle and the targeted vessel such that the shunt is downstream of the blockage and the shunt enables passage of blood from the ventricle to the targeted vessel.

12. The method of claim 11, wherein the shunt comprises a coil-shaped anchor with a central lumen.

13. The method of claim 12, wherein the shunt further comprises a plurality of perforations in the shunt to facilitate transfer of blood.

14. A method for performing cardiac artery bypass grafting, the method comprising:
navigating a catheter to a first coronary artery of a patient;
puncturing through the first coronary artery of the patient;
navigating the catheter to a second coronary artery of the patient, the second coronary artery including a blockage; and
implanting a shunt from the first coronary artery of the patient to the second coronary artery of the patient, the shunt being anchored to the second coronary artery of the patient downstream of the blockage.

15. A method for performing cardiac artery bypass grafting, the method comprising:
navigating a catheter within a targeted vessel of a patient, the targeted vessel including a blockage, the catheter navigated to a position upstream of the blockage;
puncturing through the targeted vessel with the catheter;
navigating the catheter outside the targeted vessel to a point downstream of the blockage; and
re-entering the targeted vessel with the catheter downstream of the blockage.

16. The method of claim 15, wherein the catheter comprises a coiled configuration to wrap around the targeted vessel.

17. The method of claim 15, wherein the catheter comprises a deflectable tip.

18. The method of claim 15, wherein the catheter comprises an electrocautery tip.

19. The method of claim 15 further comprising inflating a balloon to stabilize the catheter for puncturing through the targeted vessel.

20. A method for performing cardiac artery bypass grafting, the method comprising:
navigating a catheter to a targeted vessel of a patient, the targeted vessel including a blockage, the catheter navigated to a position downstream of the blockage;
anchoring an implant to the targeted vessel, the implant including anchors at a distal end of the implant configured to secure to the targeted vessel; and
creating a passageway from the implant to the targeted vessel to enable blood flow in the targeted vessel downstream of the blockage.

21. The method of claim 20, wherein the passageway is created after anchoring the implant to the targeted vessel.

22. The method of claim 20, wherein the passageway is created prior to anchoring the implant to the targeted vessel.

23. The method of claim 20, wherein creating the passageway comprises implanting a stent within the targeted vessel upstream of the blockage and anchoring a distal end of a shunt downstream of the blockage, the shunt providing the passageway between the stent and the targeted vessel at the distal end of the shunt.

24. The method of claim 23, wherein the distal end of the shunt includes a helical anchored that is anchored into muscle tissue at the targeted vessel.

25. The method of claim 20 further comprising directly visualizing the targeted vessel with a fiber optic cable that is part of the catheter.

26. The method of claim 20 further comprising visualizing angiogram dye on fluoroscopy responsive to a tip of the catheter being inserted into the targeted vessel.

27. A method for performing cardiac artery bypass grafting, the method comprising:
navigating a catheter to a targeted vessel of a patient, the targeted vessel including a blockage, the catheter navigated to a position downstream of the blockage;
anchoring a supply line that is partially embedded in a cardiac wall and partially embedded in the targeted vessel, the supply line having a diameter greater than a diameter of the targeted vessel; and
seeding angiogenesis due at least in part to the anchored supply line to provide blood flow in the targeted vessel downstream of the blockage.
